(19) United States
(12) Patent Application Publication
(10) Pub. No.: US 2024/0269486 A1
(43) Pub. Date: Aug. 15, 2024

(54) Pulsed Reduced Dose Rate Intensity Modulated Proton Therapy for Re-Irradiation of Central Nervous System Malignancies

(71) Applicant: Baptist Health South Florida, Inc, Coral Cables, FL (US)

(72) Inventors: Minesh Mehta, Coral Gables, FL (US); Alonso Gutierrez, Coral Gables, FL (US); Rupesh Kotecha, Coral Gables, FL (US)

(21) Appl. No.: 18/437,590

(22) Filed: Feb. 9, 2024

Related U.S. Application Data
(60) Provisional application No. 63/484,082, filed on Feb. 9, 2023.

(51) Int. Cl. A61N 5/10

(52) U.S. Cl. CPC A61N 5/1077; A61N 2005/1087

(57) ABSTRACT

A method for re-treatment of a tumor includes a step of providing therapy pulses to a tumor in accordance with a pulsed reduced dose rate (PRDR) therapy pattern. The therapy pulses are PRDR intensity modulated proton therapy (IMPT) pulses comprising proton pulses provided to the tumor. A system for re-treatment of a tumor is also disclosed, which includes a first sub-beam emitter for providing a first plurality of intensity modulated proton therapy (IMPT) pulses in accordance with a pulsed reduced dose rate (PRDR) therapy pattern and a second sub-beam emitter for providing a second plurality of intensity modulated proton therapy (IMPT) pulses in accordance with the PRDR therapy pattern. The system also includes a controller configured to cause the first plurality of IMPT pulses and the second plurality of IMPT pulses to be provided to the tumor sequentially with a temporal gap between delivery of the pulses.

### CROSS REFERENCE TO RELATED APPLICATION

The present application claims the benefit of U.S. Provisional Patent Application No. 63/484,082, filed Feb. 9, 2023, the disclosure of which is hereby incorporated by reference in its entirety.

### BACKGROUND OF THE INVENTION

### Field of the Invention

The present disclosure is generally directed to methods and systems for treatment of Central Nervous System (CNS) tumors and, in particular, to systems and methods for providing pulsed proton therapy for radiation and/or re-irradiation of intracranial tumors.

### Description of Related Art

Recurrent high-grade intracranial malignancies have a grim prognosis and uniform management guidelines are lacking. In particular, intracranial high-grade malignancies, such as glioblastoma—the most common malignant brain tumor in adults, are associated with frequent recurrences and poor prognosis. Salvage strategies, such as surgery, re-irradiation (reRT), systemic therapies, and Tumor Treating Fields (TTFields) have not shown significant benefit in overall survival in the recurrent setting. See Ostrom Q T, Price M, Neff C, et al. CBTRUS Statistical Report: Primary Brain and Other Central Nervous System Tumors Diagnosed in the United States in 2015-2019. Neuro-Oncology. 2022; 24 (Supplement_5): v1-v and Grossman S A, Ye X, Piantadosi S, et al. Survival of patients with newly diagnosed glioblastoma treated with radiation and temozolomide in research studies in the United States. Clin. Cancer Res. 2010; 16(8): 2443-2449.

In particular, re-irradiation is infrequently employed due to concerns regarding irreversible treatment-related toxicities. Pulsed-reduced dose rate radiotherapy (PRDR), a re-irradiation technique delivered with photons, aimed to reduce toxicity while enhancing tumor control by exploiting dose-rate effects and low-dose hyper-radiosensitivity in tumors. PRDR is also intended to facilitate sub-lethal damage repair in normal tissues. For example, PRDR photon therapy has been employed in the re-treatment of CNS malignancies to overcome dose constraints and reduce side effects associated with re-irradiation. A typical photon PRDR workflow may include providing a series of about 10 (dose) pulses of 0.2 Gy/pulse (2 Gy/fx) with an interval of 3 minutes between pulses. The series of photon pulses creates an averaged effective treatment dose rate of approximately 7 cGy/min over one treatment fraction.

However, additional treatment options for intracranial malignancies are needed, which control or reduce toxicity, while improving response rates and overall survival rate for patients. The treatment methods and medical systems of the present disclosure are intended to address such issues, which may occur using current treatment methodologies.

### SUMMARY OF THE INVENTION

According to an aspect of the disclosure, a method for re-treatment of a tumor includes a step of providing therapy pulses to a tumor in accordance with a pulsed reduced dose rate (PRDR) therapy pattern. The therapy pulses are PRDR intensity modulated proton therapy (IMPT) pulses comprising proton pulses provided to the tumor.

According to another aspect of the disclosure, a system for re-treatment of a tumor includes a first sub-beam emitter for providing a first plurality of intensity modulated proton therapy (IMPT) pulses in accordance with a pulsed reduced dose rate (PRDR) therapy pattern and a second sub-beam emitter for providing a second plurality of intensity modulated proton therapy (IMPT) pulses in accordance with the PRDR therapy pattern. The system also includes a controller electrically connected to the first and second sub-beam emitters. The controller is configured to cause the first plurality of IMPT pulses and the second plurality of IMPT pulses to be provided to the tumor sequentially with a temporal gap between delivery of one or more of the first plurality of pulses and one or more of the second plurality of pulses.

Non-limiting examples of the present invention will now be described in the following numbered clauses:

Clause 1: A method for re-treatment of a tumor, comprising: providing therapy pulses to the tumor in accordance with a pulsed reduced dose rate (PRDR) therapy pattern, wherein the therapy pulses are PRDR intensity modulated proton therapy (IMPT) pulses comprising proton pulses provided to the tumor.

Clause 2: The method of clause 1, wherein the tumor was previously treated by applying pulsed reduced dose rate (PRDR) photon therapy pulses to the tumor.

Clause 3: The method of clause 2, wherein the PRDR photon therapy pulses are about 0.2 Gy/pulse (2 Gy/fx) with an interval of at least 3 minutes between the PRDR photon therapy pulses.

Clause 4: The method of any of clauses 1-3, wherein the PRDR-IMPT pulses have an averaged effective treatment dose rate of approximately 7 cGy/min over one treatment fraction.

Clause 5: The method of any of clauses 1-4, wherein there is a temporal gap of about 4 minutes to about 5 minutes between the PRDR-IMPT pulses.

Clause 6: The method of any of clauses 1-5, wherein the PRDR-IMPT pulses are provided to repaint a treatment field previously treated by the PRDR photon therapy pulses.

Clause 7: The method of any of clauses 1-6, wherein the tumor comprises a central nervous system (CNS) tumor and/or an intracranial tumor.

Clause 8: The method of any of clauses 1-7, wherein the pulsed reduced dose rate (PRDR) therapy pattern comprises delivery of a dose of about 50 GyRBE to about 75 GyRBE divided between about 20 fractions to about 40 fractions.

Clause 9: The method of clause 8, wherein the fractions are delivered to a patient over a period of time of about 20 minutes to about 50 minutes.

Clause 10: The method of any of clauses 1-9, wherein at most about 70% of a total dose of proton therapy pulses delivered to the tumor is controlled by each beam of an ion beam applicator and a remaining at least 30% of the total dose is modulated for organs-at-risk (OAR) sparring.

Clause 11: The method of any of clauses 1-10, wherein the therapy pulses are provided according to one or more of the following therapies: stereotactic radiosurgery (SRS), fractionated SRS, hypofractionated treatment, fractionated proton therapy, or particle therapy.

Clause 12: The method of any of clauses 1-11, wherein providing the therapy pulses comprises directing a radiation beam to at least one of the following areas of a patient's body: brain, brainstem, optic chiasm, ipsilateral and/or contralateral optic nerves, ipsilateral and/or contralateral cochlea, or ipsilateral and/or contralateral hippocampus.

Clause 13: The method of any of clauses 1-12, further comprising administering an effective amount of at least one of bevacizumab and ivosidenib to a patient for treatment of the tumor.

Clause 14: A system for re-treatment of a tumor, comprising: a first sub-beam emitter for providing a first plurality of intensity modulated proton therapy (IMPT) pulses in accordance with a pulsed reduced dose rate (PRDR) therapy pattern; a second sub-beam emitter for providing a second plurality of intensity modulated proton therapy (IMPT) pulses in accordance with the PRDR therapy pattern; and a controller electrically connected to the first and second sub-beam emitters, wherein the controller is configured to cause the first plurality of IMPT pulses and the second plurality of IMPT pulses to be provided to the tumor sequentially with a temporal gap between delivery of one or more of the first plurality of IMPT pulses and one or more of the second plurality of IMPT pulses.

Clause 15: The system of clause 14, wherein the controller is configured to cause the first sub-beam emitter and the second sub-beam emitter to provide PRDR-IMPT pulses having an averaged effective treatment dose rate of approximately 7 cGy/min over one treatment fraction.

Clause 16: The system of clause 14 or clause 15, wherein the temporal gap between the delivery of one or more of the first plurality of IMPT pulses and one or more of the second plurality of IMPT pulses is from about 4 minutes to about 5 minutes.

Clause 17: The system of any of clauses 14-16, wherein the tumor is a central nervous system (CNS) tumor and/or an intracranial tumor.

Clause 18: The system of any of clauses 14-17, wherein at most about 70% of a total dose provided by the first sub-beam emitter and the second sub-beam emitter is controlled by the sub-beam emitters, and a remaining at least 30% of the total dose is modulated for organs-at-risk (OAR) sparring.

Clause 19: The system of any of clauses 14-18, wherein the controller is configured to cause the first and second sub-beam emitters to deliver the first and second plurality of IMPT pulses for a period of time of about 20 minutes to about 50 minutes in order to provide a total dose of the IMPT pulses to the tumor. Clause 20: The system of any of clauses 14-19, wherein the controller is configured to cause the first sub-beam emitter and the second sub-beam emitter to deliver the pulsed reduced dose rate (PRDR) therapy pattern, which comprises delivery of a total dose of from about 50 GyRBE to about 75 GyRBE divided over about 20 fractions to about 40 fractions.

### BRIEF DESCRIPTION OF THE DRAWINGS

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limit of the invention.

FIG. 1A is a schematic drawing of a PRDR-IMPT delivery strategy, according to an aspect of the present disclosure;

FIG. 1B is another schematic drawing of a PRDR-IMPT delivery strategy, according to an aspect of the present disclosure;

FIG. 2 is a medical image that provides an isodose comparison of PRDR-IMRT and IMPT plans;

FIG. 3 is a line graph showing a dose-volume histogram (DVH) comparison between PRDR-IMRT and PRDR-IMPT plans for a sample subject;

FIGS. 4A-4D are line graphs comparing an uninvolved brain V10Gy, V20Gy, V30Gy and V40Gy for PRDR photon and PRDR-IMPT plans as a function of target (PTV) size;

FIGS. 5A and 5B are histograms showing minimum local and global gamma analysis for patients;

FIG. 6 is a table showing a global gamma analysis for repainted and non-repainted cases;

FIGS. 7A and 7B are tables listing outcomes for recurrent high-grade gliomas with systemic therapies on prospective trials;

FIG. 8 is a line graph showing results for a cognitive battery performed for a patient;

FIG. 9 is a flow chart showing steps of a method for re-treatment of a tumor, according to an aspect of the present disclosure;

FIG. 10 is a schematic drawing of a system for re-treatment of a tumor, according to an aspect of the present disclosure;

FIGS. 11A and 11B are tables listing characteristics of five treated patients describing initial diagnoses, first course of radiation therapy characteristics and reRT (TMPPR) characteristics;

FIG. 12A shows medical images for patients that have undergone multiple courses of radiotherapy;

FIG. 12B is a table listing information for courses of doses and cumulative doses of PRDR-IMPT provided to patients; and

FIG. 12C shows medical images for a follow-up MRI performed on a patient four weeks after PRDR-IMPT treatment.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention. Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “comprising,” “comprise” or “comprised,” and variations thereof, are meant to be open ended. As used herein, the term “patient” or “subject” refers to members of the animal kingdom including but not limited to human beings.

The present disclosure is directed to a treatment methodology, referred to by the present inventors as a temporally modulated pulsed proton re-irradiation (TMPPR) therapy, for treatment of intracranial malignant tumors. The methodology involves delivery of pulsed-reduced dose rate (PRDR) radiotherapy for re-irradiation of CNS tumors. The methodology also involves steps for dosimetric treatment planning evaluation to investigate potential dose delivery benefits with intensity-modulated proton therapy (IMPT). To develop these methodologies, the present inventors evaluated whether PRDR-IMPT offers a dosimetric advantage over the photon PRDR plans described previously. Based on such evaluations, the present inventors have determined that proton therapy (PT) has an overall dosimetric benefit over photon therapy in CNS malignancies. In particular, IMPT has been dosimetrically demonstrated to result in lower maximum, average, and median doses to critical substructures of the brain compared to passive scatter proton therapy or intensity-modulated radiotherapy (IMRT) delivered using photons, as shown by the examples provided herein.

In some examples, the methods of the present disclosure include providing therapy pulses to the tumor in accordance with a pulsed reduced dose rate (PRDR) therapy pattern. The therapy pulses can be PRDR intensity modulated proton therapy (IMPT) pulses comprising proton pulses provided to the tumor. In some examples, the PRDR-IMPT pulses can have an averaged effective treatment dose rate of approximately 7 cGy/min over one treatment fraction. In some examples, the plurality of PRDR-IMPT pulses can be provided to a treatment field previously treated by the PRDR photon therapy pulses (e.g., photon therapy pulses of about 0.2 Gy/pulse (2 Gy/fx) with an interval of at least 3 minutes between pulses).

In some examples, reRT can be delivered to patients according to a fractionated radiosurgery or hypofractionated therapy schedule, such as by providing a total dose (30-50 Gy) over a predetermined number of fractions (e.g., from 10 fractions to 30 fractions). However, limited randomized data is available to support use of reRT for pulsed radiation therapy. In fact, the best data to date comes from the RTOG 1205 trial, which compared bevacizumab alone with radiotherapy [35 Gy in 10 fractions] and showed a benefit in 6-month progression-free survival (PFS) from 29% to 52%. However, no benefit in overall survival was observed. See Tsien C I, Pugh S L, Dicker A P, et al. NRG Oncology/RTOG1205: A Randomized Phase II Trial of Concurrent Bevacizumab and Reirradiation Versus Bevacizumab Alone as Treatment for Recurrent Glioblastoma. J Clin Oncol. 2022: Jco2200164.

Nevertheless, in developing the treatment methodologies disclosed herein, the present inventors have come to believe that of available treatment options for intracranial malignancies, re-irradiation therapy (reRT) remains underutilized given the potential for irreversible treatment-related toxicities using other treatment methods. In particular, because tumors typically recur within or adjacent to the radiotherapy field, re-irradiation therapy will generally result in direct overlap with a previously treated region(s) of the brain. Accordingly, re-irradiation therapy may, in fact, reduce overcall toxicity effects compared to other treatment methods.

It has also been determined that systemic therapies alone in the recurrent setting are associated with poor (<10%) response rates. See Ellingson B M, Wen P Y, Chang S M, et al. Objective response rate (ORR) targets for recurrent glioblastoma clinical trials based on the historic association between ORR and median overall survival. Neuro Oncol. 2023. Furthermore, although no prospective randomized evidence supports one approach over another, a secondary analysis of Radiation Therapy Oncology Group (RTOG) 0525 observed a benefit to reRT, although it was limited in scope as few patients underwent a second course of radiotherapy (4% reRT alone and 10% with systemic therapy). See Shi W, Scannell Bryan M, Gilbert M R, et al. Investigating the Effect of Reirradiation or Systemic Therapy in Patients With Glioblastoma After Tumor Progression: A Secondary Analysis of NRG Oncology/Radiation Therapy Oncology Group (RTOG) Trial 0525. Int J Radiat Oncol Biol Phys. 2018; 100(1):38-44.

### Pulsed-Reduce Dose Rate Therapy and Intensity-Modulated Proton Therapy

Despite the lack of previously available randomized data supporting use, the present inventors believe that pulsed-reduced dose rate (PRDR) radiotherapy delivered with protons can be an effective reRT technique, which reduces toxicity after prior radiation therapy in recurrent/progressive disease in patients with central nervous system (CNS) tumors. In some examples, PRDR can comprise delivering radiotherapy subfractions at specific time intervals within a single fraction. While not intending to be bound by theory, it is believed that there are two radiobiological advantages to this technique. First, the technique provides a low-dose hyper-radiosensitivity of proliferative tumor cells irradiated at doses less than 0.5 Gy. Second, the technique provides non-proliferative normal tissue low-dose rate hypo-radiosensitivity, thereby increasing the therapeutic index as tumor kill is increased and promoting sub-lethal damage repair occurring in normal cells reduces toxicities.

Such radiobiological advantages are also identified in prior studies. In 2007, Cannon et al., documented their clinical experience in treating a recurrent brain tumor with PRDR, showing a dramatical radiographic response and a clinical improvement associated with no toxicity. See Cannon G, Tomé W, Robins H, Howard S. Pulsed reduced dose-rate radiotherapy: case report: a novel re-treatment strategy in the management of recurrent glioblastoma multiforme. Journal of neuro-oncology. 2007; 83(3). In reviewing recent usage statistics for photon PRDR in patients with recurrent primary CNS malignant tumors, it was also observed that a median PRDR dose was 52 Gy (22 to 60 Gy) with a median cumulative dose of 110.3 Gy delivered to a median tumor volume of 369.1 cc. After a median follow-up of 8.7 months, 67% of patients had grade 2+ treatment-related toxicitics. See Kutuk T, Tolakanahalli R, McAllister N, et al. Pulsed-Reduced Dose Rate (PRDR) Radiotherapy for Recurrent Primary Central Nervous System Malignancies: Dosimetric and Clinical Results. Cancers. 2022; 14(12).

As previously described, the present inventors believe that proton therapy (PT) has overall dosimetric benefit over photon therapy in CNS malignancies. The latest form of PT, intensity-modulated proton therapy (IMPT), has been dosimetrically demonstrated to result in lower maximum, average, and median doses to critical substructures of the brain compared to passive scatter proton therapy or intensity-modulated radiotherapy (IMRT) delivered using photons. In particular, in a recent study, the present inventors determined that IMPT was able to spare the uninvolved brain (V_{20Gy}) and a reduction on D_{0.03cc} to the brain and optic chiasm when compared to IMRT. In patients undergoing reRT, these reduced doses to critical substructures can decrease risks for treatment-related toxicities, allowing the ability to deliver a definitive dose.

However, despite the potential decreased risks of reRT, the present inventors have recognized that intracranial reRT presents a challenging situation for which no consensus exists regarding cumulative dose evaluation, or for optimal dose and fractionation. In an effort to reduce these photon PRDR toxicities, the present inventors have postulated that pulsed-reduced doses delivered with protons can be an alternative for these groups of patients due to the reduced low and intermediate doses afforded by proton therapy to the surrounding (often previously treated) tissues.

Current guidelines consider reRT as a possible approach, among other options, as a local treatment for recurrent glioblastoma. In some examples reRT can be delivered using a variety of techniques, from stereotactic radiosurgery (SRS), fractionated SRS, hypofractionated treatment, conventionally fractionated photon therapy, or even particle therapy. SRS, supported by multiple retrospective datasets, has been traditionally delivered to recurrent limited target volumes (4-10 cc). Furthermore, multiple retrospective and prospective series have investigated fractionated SRS (FSRS) or hypofractionated schedules for recurrent glioblastoma, where the target volumes were fairly modest at 8.5-34 cc (FSRS) and 33-145 cc (hypofractionated reRT). Conventionally fractionated reRT has also been used (most commonly 36 Gy in 18 fractions) for larger volume recurrences and associated with lower rates of radiation necrosis when compared to more hypofractionated schedules (including SRS/FSRS). Taking into account the large treatment volumes involved in patients (range: 109.1-442.6 cc), which are too large for hypofractionation, and the close anatomical relation to critical structures, the present inventors have recognized benefits of combining the radiobiological benefit of delivering TMPPR with the use of protons to deliver meaningful dose to the tumor, while sparing the remainder of the uninvolved brain.

As shown by the results for Example 2, which is described in the Examples section of this disclosure, promising responses were seen for patients (1 complete response and 3 partial responses) treated by TMPPR. One of the patients achieved a complete response and at a follow-up showed a clinical improvement associated with imaging findings of a regression of all enhancing and a significant component of the non-enhancing disease (T1 enhancement and associated FLAIR infiltration), as well as resolution of midline shift and mass effect on the adjacent brain parenchyma and the absence of a prior elevated cerebral blood volume (rCBV) on the perfusion MRI. At a follow-up 5 months after the TMPPR therapy, the patient developed leptomeningeal failure in the contralateral untreated brain (previously no disease). Interestingly, a second patient treated with TMPPR also exhibited a partial response to treatment at the area of TMPPR in the right frontal lobe. However, the patient also has leptomeningeal failure on the contralateral brain on longer follow-up.

Overall, three of the patients in Example 2 partially respond to TMPPR and those patients received systemic therapy peri-RT as well. These outcomes resulted in a high overall response rate not seen in prior studies with other strategies of treatment. Furthermore, the results of clinical trials for systemic therapies in high-grade glioma patients at first recurrence or progression have shown varying levels of success, with the objective response rate (ORR) ranging from 5% to 41.5%. However, substantial responses or complete responses to these treatments are uncommon, with none being observed for lomustine alone and only 2.4% for the combination of bevacizumab and irinotecan. When comparing bevacizumab versus nivolumab, Reardon et al. found, for bevacizumab 2.6% complete response and 20.5% partial response. See Reardon D A, Brandes A A, Omuro A, et al. Effect of Nivolumab vs Bevacizumab in Patients With Recurrent Glioblastoma: The CheckMate 143 Phase 3 Randomized Clinical Trial. JAMA Oncol. 2020; 6(7): 1003-1010. Moreover, an ORR of only 35% (CR in one case and 17 cases of PR's) was seen in another study with bevacizumab alone.

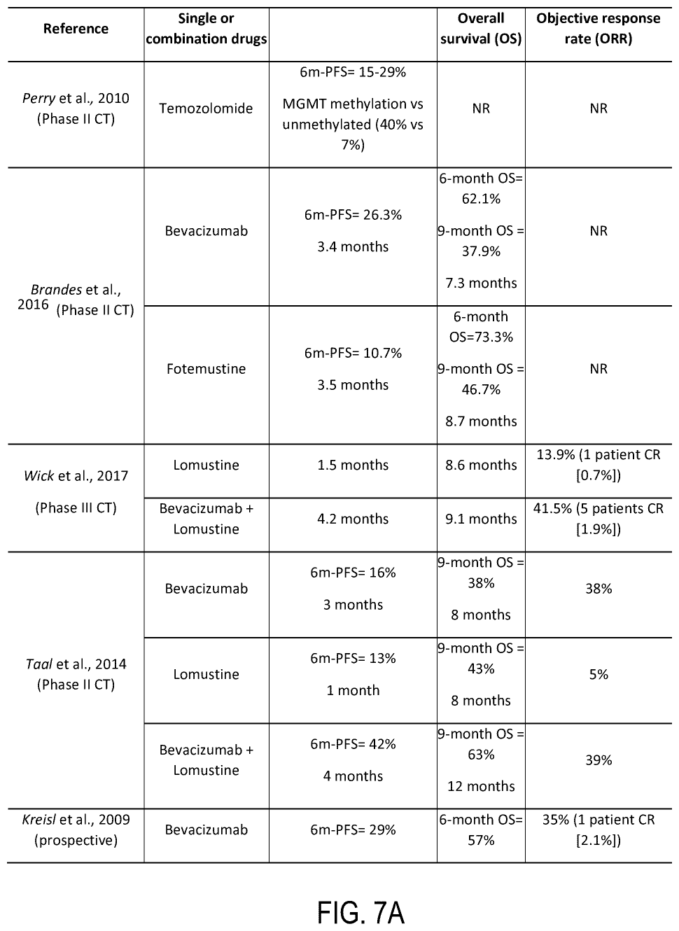

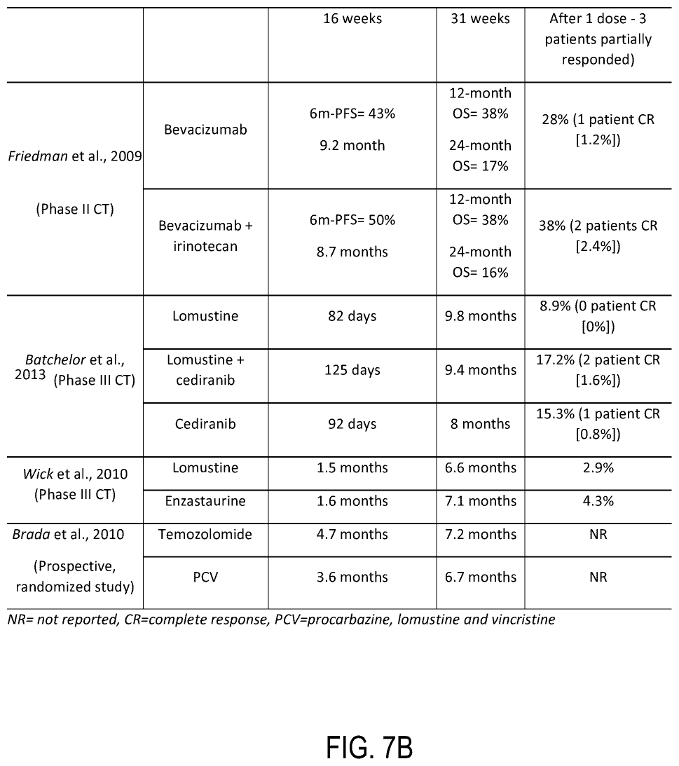

Novel systemic therapies have been studied in clinical trial settings with modest activity in high-grade gliomas, as shown by the tables in FIGS. 7A and 7B. Specifically, the tables in FIGS. 7A and 7B list outcomes for recurrent high-grade gliomas with systemic therapies on prospective trials for the following clinical studies: Perry J R, Bélanger K, Mason W P, et al. Phase II trial of continuous dose-intense temozolomide in recurrent malignant glioma: RESCUE study, J Clin Oncol. 2010; 28(12):2051, 2057; Brada M, Stenning S, Gabe R, et al. Temozolomide versus procarbazine, lomustine, and vincristine in recurrent high-grade glioma, J Clin Oncol. 2010; 28(30):4601-4608; Wick W, Gorlia T, Bendszus M, et al. Lomustine and Bevacizumab in Progressive Glioblastoma, N Engl J Med. 2017; 377(20): 1954-1963; Taal W, Oosterkamp H M, Walenkamp A M, et al. Single-agent bevacizumab or lomustine versus a combination of bevacizumab plus lomustine in patients with recurrent glioblastoma (BELOB trial): a randomized controlled phase 2 trial, Lancet Oncol. 2014; 15(9):943-953; Kreisl T N, Kim L, Moore K, et al. Phase II trial of single-agent bevacizumab followed by bevacizumab plus irinotecan at tumor progression in recurrent glioblastoma, J Clin Oncol. 2009; 27(5):740-745; Friedman H S, Prados M D, Wen P Y, et al. Bevacizumab alone and in combination with irinotecan in recurrent glioblastoma, J Clin Oncol. 2009; 27(28):4733-4740; Batchelor T T, Mulholland P, Neyns B, et al. Phase III randomized trial comparing the efficacy of cediranib as monotherapy, and in combination with lomustine, versus lomustine alone in patients with recurrent glioblastoma, J Clin Oncol. 2013; 31(26):3212-3218; Wick W, Puduvalli V K, Chamberlain M C, et al. Phase III study of enzastaurin compared with lomustine in the treatment of recurrent intracranial glioblastoma, J Clin Oncol. 2010; 28(7): 1168-1174; and Brada M, Stenning S, Gabe R, et al. Temozolomide versus procarbazine, lomustine, and vincristine in recurrent high-grade glioma, J Clin Oncol. 2010; 28(30):4601-4608.

Recently, the Checkmate 143 trial, comparing the use of nivolumab vs. bevacizumab at first recurrence did not demonstrate a benefit in OS (9.8 vs. 10 months); moreover, the ORR was higher in bevacizumab arm (7.8 vs. 23.1%), with a complete response observed in only 2 of 153 (1.3%) patients receiving nivolumab. Also, the combination of nivolumab with ipilimumab did not show any complete response (CR) and almost no difference with nivolumab alone for PR (2 versus 1, respectively).

Recently, Ellingson et al. described the following objective response rates (ORRs) for different therapies from past clinical trials (68 treatments arms; 4793 patients) for recurrent high-grade gliomas (≤3 recurrences, but great majority were first recurrences): chemotherapy 6.1%, biological agents 3.37%, immunotherapies 7.97%, and antiangiogenics 26.8%. See Ellingson B M, Wen P Y, Chang S M, et al. Objective response rate (ORR) targets for recurrent glioblastoma clinical trials based on the historic association between ORR and median overall survival. Neuro Oncol. 2023. The combination of data from chemotherapy, biologics, and immunotherapy suggested a strong correlation between ORR and median overall survival (OS), with ORR values greater than 25% leading to a median OS of over 15 months.

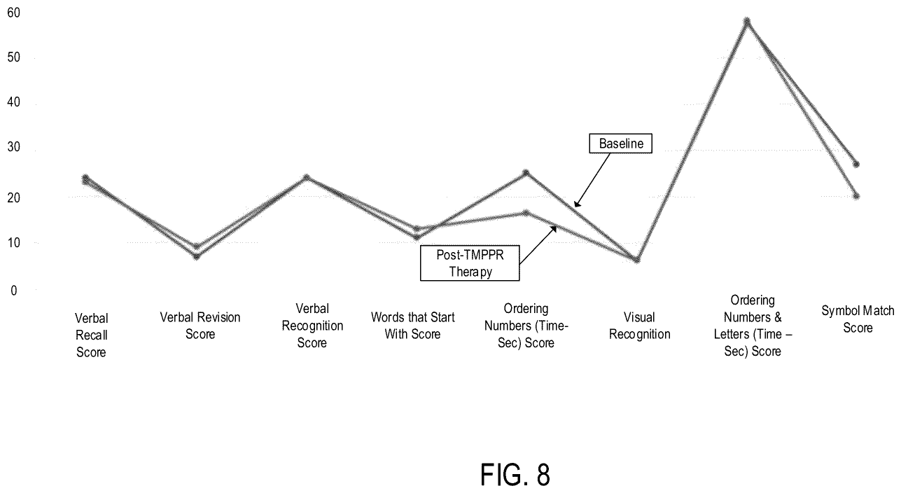

Neurocognitive function decline is one of the major concerns for brain irradiation, especially for patients with multiply recurrent tumors who undergo surgery, multiple lines of systemic therapy, and reRT. In Example 2 of the present disclosure, the inventors objectively tested one patient with baseline (pre-TMPPR) and follow-up (four week after TMPPR) cognitive batteries, as shown in FIG. 8, with preservation across multiple neurocognitive domains. Given the ability of proton therapy (PT) to reduce doses to the surrounding normal structures, such as hippocampus and temporal lobes (which are organs associated with neurocognition), it is believed that protection of such normal tissue represents a potential advantage of this technique. Prospective data have shown that proton therapy is capable of preserving neurocognitive function in patients with low-grade gliomas, compared to historical controls.

The present inventors do recognize, however, that there remains a lack of consensus regarding reRT to the brain in terms of tolerance of critical organs and the potential for debilitating or even fatal effects. For example, Mayer et al. conducted an overview based on clinical data, by comparing several prior studies on the tolerance of the brain to reRT for gliomas. They found an increased risk for radiation necrosis when the equivalent dose in 2-Gy fractions (EQD2) was over 100 Gy. It is important to note that cumulative doses to the normal brain were only reported, not considering the total target volumes involved. See Mayer R, Sminia P. Reirradiation Tolerance of the Human Brain. International Journal of Radiation Oncology*Biology*Physics. 2008; 70(5):1350-1360. Stiefel et al. conducted a retrospective analysis in 76 patients (including primary tumors and metastasis) with at least two prior courses of radiotherapy. For patients exceeding the D_{0.1cc} of 100 Gy (EQD2) to the brain, the median D_{0.1cc} was 114 Gy (range 100-161.5) and resulted in only 2 cases of high grade (>grade 3) toxicity. Also, they concluded that keeping cumulative doses to the brain up to 120 Gy EQD2, below 100 Gy for brainstem and below 75 Gy EQD2 to chiasm and optic nerves was safc. See Stiefel I, Schröder C, Tanadini-Lang S, et al. High-dose re-irradiation of intracranial lesions Efficacy and safety including dosimetric analysis based on accumulated EQD2Gy dose EQD calculation. Clin Transl Radiat Oncol. 2021; 27:132-138. Furthermore, prior studies of photon PRDR have traditionally limited the dose of the brainstem or optic chiasm to 50 Gy in the re-irradiation, regardless the dose received in the prior course of radiotherapy.

Despite such lack of consensus, the present inventors believe that the methods of the present disclosure are shown to provide a therapeutic benefit with low levels of toxicity. In particular, considering the large volumes of reRT (median CTV of 298 cc), and the high median maximal cumulative doses to critical structures, such as the brain [D_{0.5cc}=116.3 Gy(RBE), and D_{1cc}=115.8 Gy(RBE)], brainstem [D_{0.5cc}=100.3 Gy(RBE), and D_{1cc}=92.6 Gy(RBE)], optic chiasm [D_{0.03cc}=65.9 Gy(RBE)], and ipsilateral optic nerve [D_{0.03cc}=67.2 Gy(RBE)], the methods of the present disclosure have been able to deliver an appropriate definitive dose without any severe treatment-related acute toxicity (alopecia grade 2 and one case of radiation necrosis grade 2).

### Treatment Methods Using PRDR and IMPT

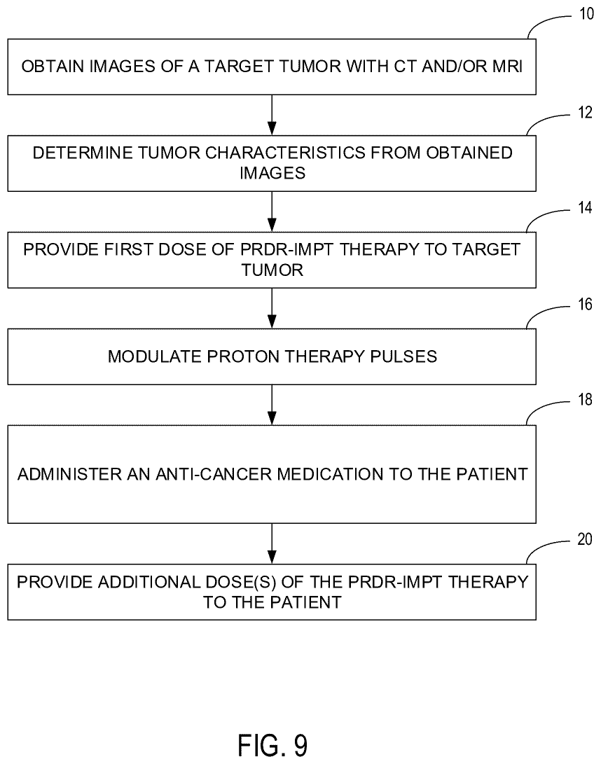

FIG. 9 is a flow chart showing steps of a method for re-treatment of a tumor, which includes features of the pulsed-reduced dose rate therapy and intensity-modulated proton therapy of the present disclosure.

At step 10, the method comprises a step of imaging a target tumor. For example, imaging comprise performing a computerized tomography (CT) scan of a target arca of a patient's body (e.g., performing a CT scan using 1-mm slice thickness). Imaging can also comprise diagnostic magnetic resonance (MRI), such as MRI imaging including T1 post-Gadolinium (Gd) and T2-weighted fluidattenuated inversion recovery (T2/FLAIR) sequences.

The method further comprises a step 12 of detecting or identifying a tumor for treatment in captured medical images, such as a central nervous system tumor and/or intracranial tumor. In some examples, the identified tumor can be a tumor that was previously treated by applying pulsed reduced dose rate (PRDR) photon therapy pulses to the tumor. For example, the photon therapy may have been applied as a series of discrete pulses, such as pulses of about 0.2 Gy/pulse (2 Gy/fx) with an interval of at least 3 minutes between pulses.

At step 14, the method can further comprise providing a first dose of therapy pulses to the identified tumor in accordance with a pulsed reduced dose rate (PRDR) therapy pattern. In some examples, the therapy pulses can be provided according to one or more of the following therapies: stereotactic radiosurgery (SRS), fractionated SRS, hypofractionated treatment, fractionated proton therapy, or particle therapy. Furthermore, in some examples, providing the therapy pulses can comprise directing radiation (e.g., proton) beams to at least one of the following areas of a patient's body: brain, brainstem, optic chiasm, ipsilateral and/or contralateral optic nerves, ipsilateral and/or contralateral cochlea, or ipsilateral and/or contralateral hippocampus of a patient. As previously described, the therapy pulses are PRDR intensity modulated proton therapy (IMPT) pulses comprising proton pulses. In some example, the therapy pulses are provided to a tumor, which has previously been treated by photon therapy pulses. In such instances, the PRDR-IMPT pulses can be provided to, in effect, repaint a treatment field previously treated by the PRDR photon therapy pulses.

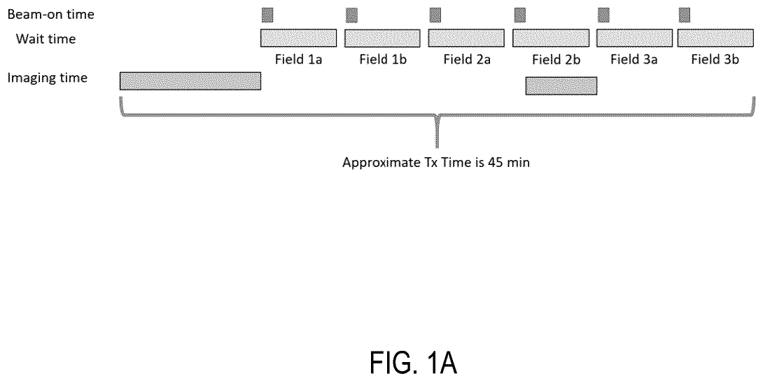

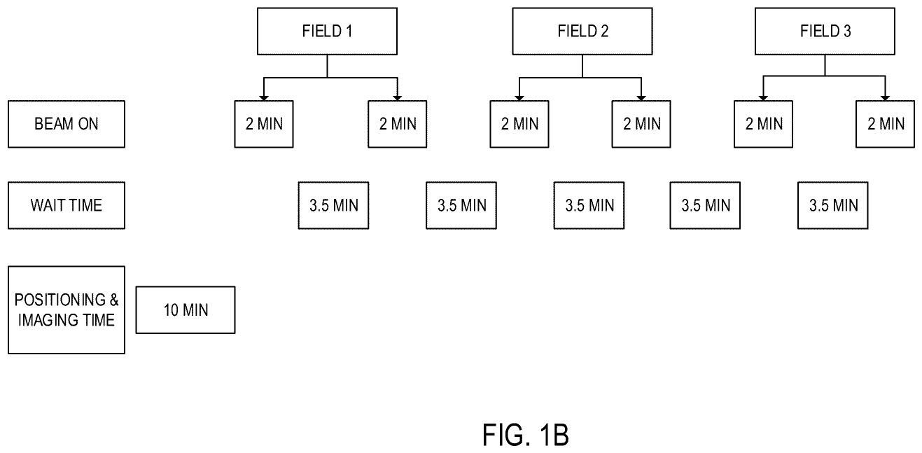

In some examples, the PRDR-IMPT pulses can be delivered as fractions or partial doses which, over time, accumulate to the full dose amount. In particular, as shown in FIGS. 1A and 1B, a dose pattern or strategy can include applying brief pulses (shown by the “beam-on time” in FIGS. 1A and 1B) followed by longer “wait time(s)” between the pulses. In some examples, the pulses can have an averaged effective treatment dose rate of approximately 7 cGy/min over one treatment fraction. The fractions or pulses can be separated by a time gap, such a gap of about 1 minutes to about 10 minutes or about 4 minutes to about 5 minutes. In some preferred examples, the pulsed reduced dose rate (PRDR) therapy pattern can comprise delivery of a total dose of about 50 GyRBE to about 75 GyRBE divided between about 20 fractions to about 40 fractions. In some examples, delivery of the total dose can occur over a period of time of about 20 minutes to about 50 minutes or, as shown in FIG. 1A about 45 minutes or FIG. 1B about 39.5 minutes.

At step 16, in some examples, the method can further comprise, after delivery of a portion of a total dose of the PRDR-IMPT pulses, modulating the proton therapy pulses. In particular, a pulse rate or intensity can be modulated for organs-at-risk (OAR) sparring and/or for protection of other normal tissue proximate to a tumor site. In some examples, at most about 70% of a total dose of proton therapy pulses delivered to the tumor can be controlled by each beam of an ion beam applicator and a remaining at least 30% of the total dose can be modulated for OAR sparring.

At step 18, the method can further comprise administering an anti-cancer and/or chemotherapy therapeutic agent to a patient. For example, the method can comprise administering an effective amount of a therapeutic agent for cancer therapy, such as an anti-cancer medication, chemotherapy agent, immunotherapy agent, and/or biological therapy agent to the patient. In some examples, at least one of bevacizumab or ivosidenib can be administered to the patient for treatment of the tumor. In some examples, the anti-cancer therapeutic agent can be delivered to the patient prior to beginning intensity modulated proton therapy and/or before the patient receives pulsed reduced does rate photon therapy. In other examples, treatment with the PRDR-IMPT treatment and the treatment with the therapeutic agent can occur together. For example, the patient may receive a dose or the therapeutic agent soon before or shortly after receiving the PRDR-IMPT therapy.

At step 20, the method can comprise providing additional doses of the therapy pulses to the identified tumor in accordance with the pulsed reduced dose rate (PRDR) therapy pattern. For example, a patient may receive a dose of the therapy pulses daily for a period of days or weeks. In other examples, the patient may receive a dose of the therapy pulses several times each day, such as every 2 hours, 4 hours, 6 hours, or 12 hours. As discussed previously, the patient may also receive doses of the therapeutic agent along with receiving the doses of the therapy pulses.

### Treatment Systems for PRDR-IMPT Therapy

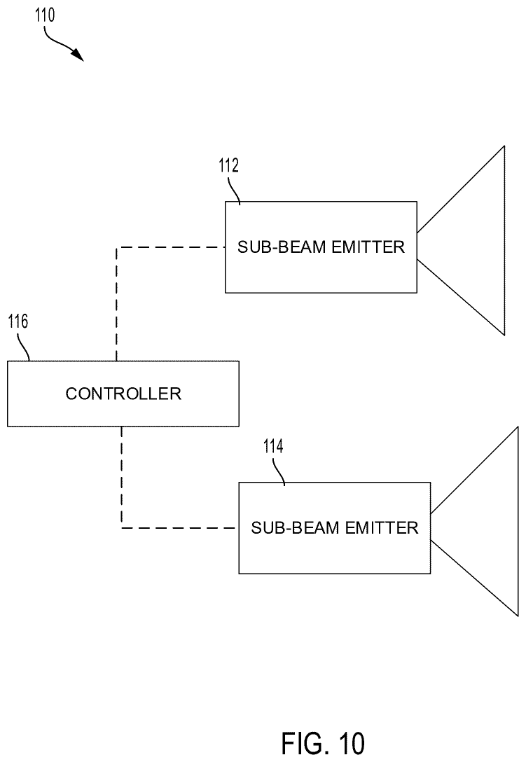

FIG. 10 is a schematic drawing of a system for re-treatment of a tumor, which includes features of the pulsed-reduced dose rate therapy and intensity-modulated proton therapy of the present disclosure.

As shown in FIG. 10, the system 110 comprises a first sub-beam emitter 112 for providing a first plurality of intensity modulated proton therapy (IMPT) pulses in accordance with a pulsed reduced dose rate (PRDR) therapy pattern and a second sub-beam emitter 114 for providing a second plurality of intensity modulated proton therapy (IMPT) pulses in accordance with the PRDR therapy pattern. The system further comprises a controller 116, such as a portable computer or microprocessor, electrically connected to the first and second sub-beam emitters 112, 114. The controller 116 can be configured to cause the first plurality of IMPT pulses and the second plurality of IMPT pulses to be provided to the tumor sequentially with a temporal gap, which can be about 1 minute to about 10 minutes or about 4 minutes to about 5 minutes in duration, between delivery of one or more of the first plurality of pulses and one or more of the second plurality of pulses. As previously described, the system 110 can be used for treatment of central nervous system (CNS) malignancies or tumors, such as intracranial tumors. Also, the plurality of PRDR-IMPT pulses provided to the CNS tumor can have an average dose rate of 7 cGy/min over a treatment fraction.

In some examples, the controller 116 can also be configured to control modulation of the pulses. For example, the controller 116 can be configured to modify the pulses such that at most about 70% of a total dose provided by the first sub-beam emitter 112 and the second sub-beam emitter 114 is controlled by the sub-beam emitters 112, 114, and a remaining at least 30% of the total dose is modulated for organs-at-risk (OAR) sparring.

In some examples, the controller 116 can also be configured to control a duration and/or frequency of pulses in accordance with the PRDR therapy pattern, such that a full or total dose of therapy pulses is provided to the tumor within a reasonable period of time. For example, the controller 116 can be configured to cause the first and second emitters 112, 114 to deliver the first and second plurality of pulses for a period of time of about 20 minutes to about 50 minutes in order to provide a total dose of the IMPT pulses to the tumor. In a particular example, the controller 116 can be configured to cause the first sub-beam emitter 112 and the second sub-beam emitter 114 to deliver the pulsed reduced dose rate (PRDR) therapy pattern, which comprises delivery of a total dose of from about 50 GyRBE to about 75 GyRBE divided over about 20 fractions to about 40 fractions. In some examples, the full dose of about 20 fractions to about 40 fractions can be delivered within a period of time of about 20 minutes to about 50 minutes.

### EXAMPLES

The following examples are presented to demonstrate the general principles of the invention. The invention should not be considered as limited to the specific examples presented.

### Examples 1.1-1.3

### Materials and Methods

An exemplary photon PRDR workflow consists of 10 (dose) pulses of 0.2 Gy/pulse (2 Gy/fx) with an interval of 3 minutes between pulses. This workflow creates an averaged effective treatment dose rate of approximately 7 cGy/min over one treatment fraction. To generate a similar averaged effective dose rate with IMPT, a treatment field was broken into two sub-beams, which were delivered sequentially with a temporal gap between deliveries tailored to achieve an average dose rate of 7 cGy/min over a treatment fraction. With most CNS cases treated commonly with three equally weighted IMPT beams to 1.8 Gy/fx, each treatment field delivers 0.6 Gy per fraction to the target tumor. If each field is sub-divided into two deliverable sub-beams combined with a temporal gap of about 4 minutes to about 5 minutes between sub-beams, an averaged treatment dose rate of 7 cGy/min over the treatment fraction is possible.

In Example 1.1, to evaluate the dosimetric parameters of IMPT-PRDR, fifteen (15) previously treated IMRT-PRDR CNS cases were comparatively planned. IMRT plans were planned to a planned target volume (PTV) (3 mm expansion of the clinical target volume (CTV)), while IMPT plans were robustly optimized to the CTV with a 3 mm setup uncertainty and 3.5% range uncertainty. IMRT and IMPT plans were normalized such that 95% of the PTV received 100% of the prescription dose.

In Example 1.2, six (n=6) previously treated photon PRDR patients were randomly selected and retrospectively re-planned for IMPT using standard treatment protocols (e.g., a 3 mm dose grid and a modified SFO approach with 3 or 4 treatment fields). 70% of the total dose was controlled by each individual beam, while a remaining 30% was allowed to modulate in order to help spare critical OARs and normal brain tissue. IMPT and photon plans were normalized to 95% at Rx for PTVs coverage for comparison. The PRDR-IMPT treatment strategy was used to repaint each treatment field once in order to achieve an averaged effective treatment dose rate of approximately 7 cGy/min over one treatment fraction. To quantify dose accuracy of non-repainted vs. the repainted proton plans, measurements were completed using an IBA Matrixx 2D Array Detector in a DigiPhant PT water phantom (right). All treatment fields were evaluated at a common depth in water of 3 cm.

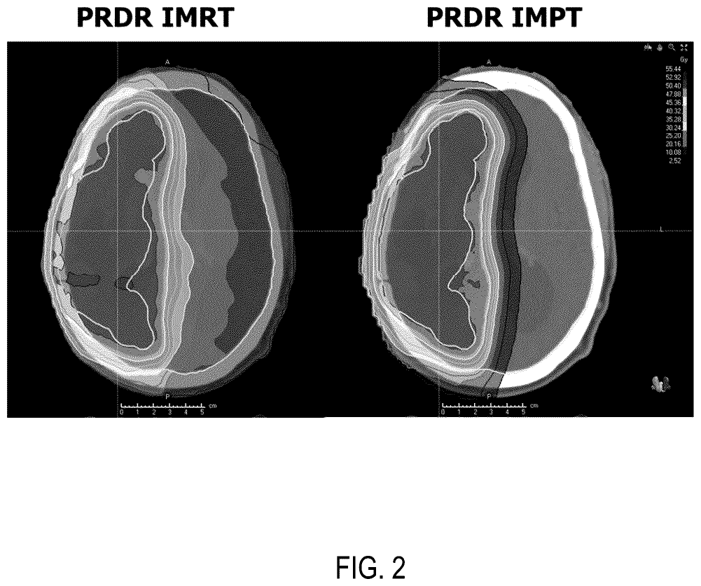

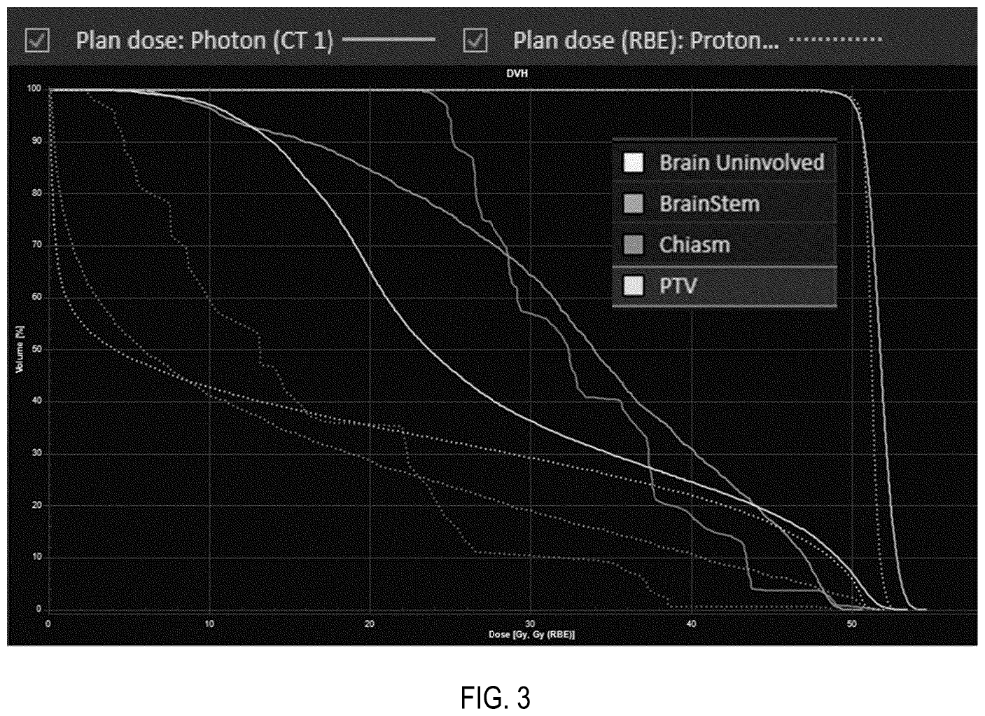

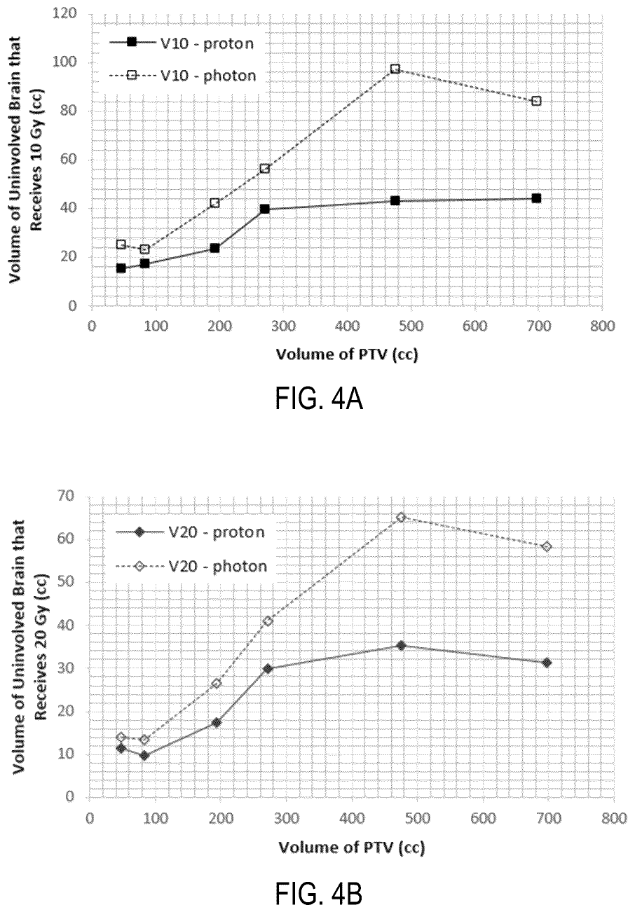

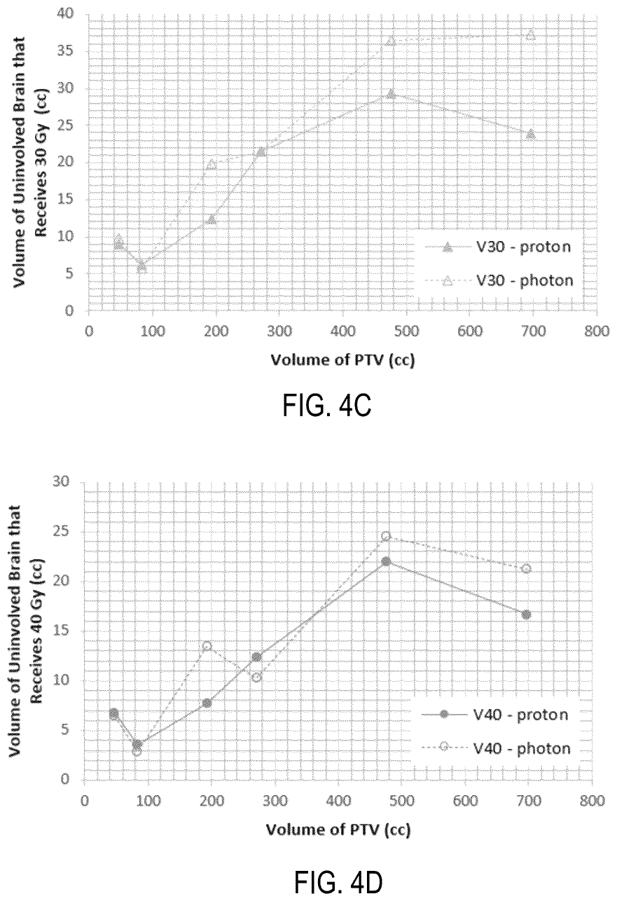

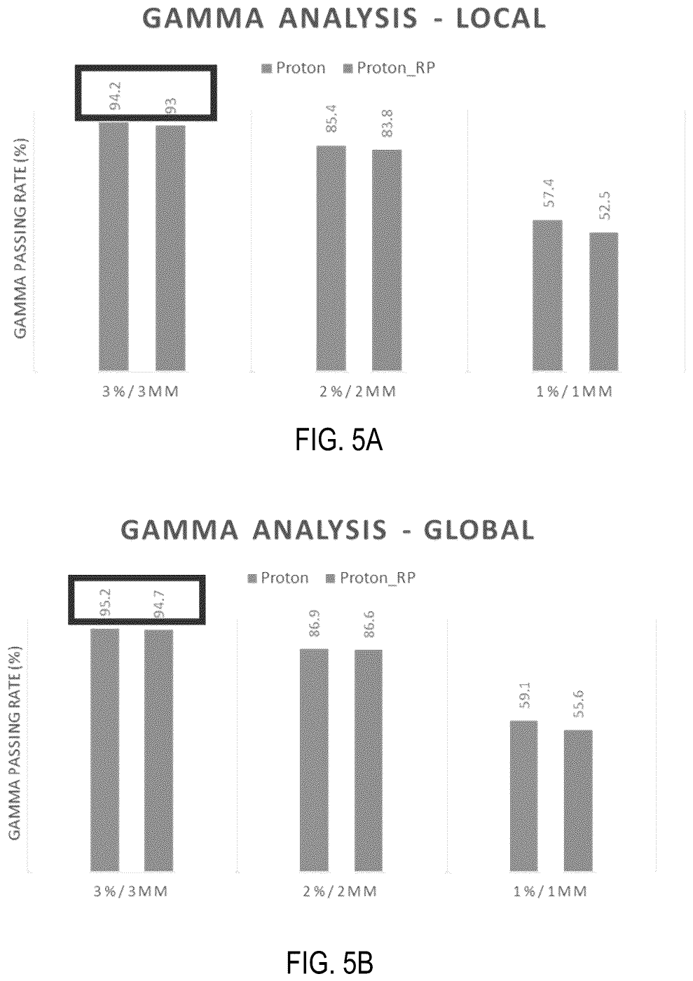

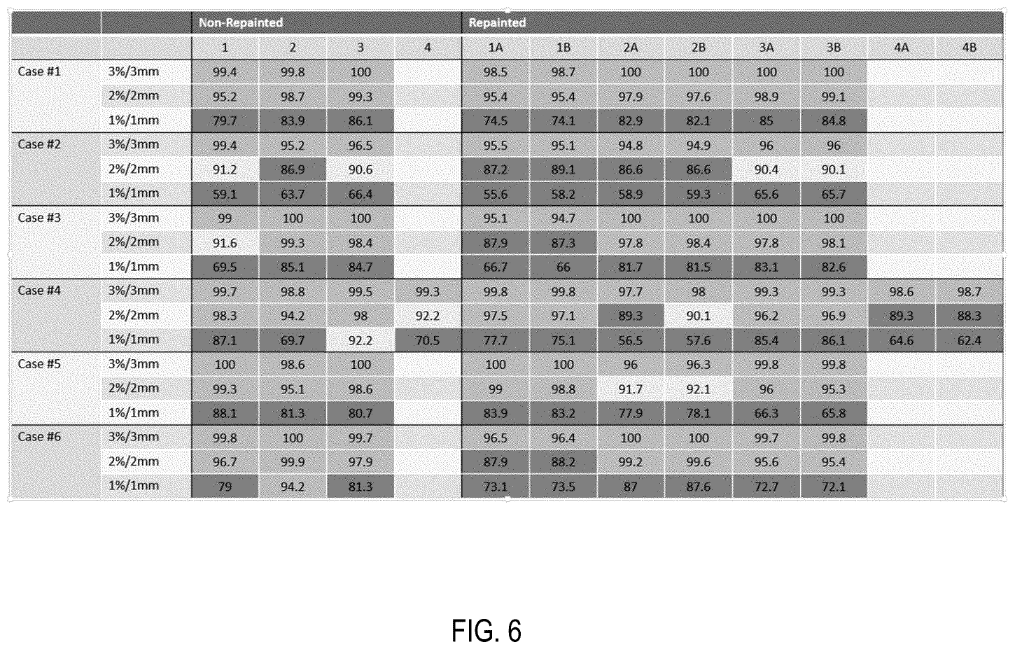

Measurements results were analyzed using the IBA software, myQA. Global gamma criteria of 3%/3 mm>93% (clinical standard) were employed to determine clinical acceptability. Additionally, local and global gamma analysis using 2%/2 mm and 1%/1 mm criteria were also completed. Results for each of the six cases are provided in Table 1, which is shown below, as well as in FIGS. 2-6. In particular, FIG. 2 shows medical images that provide an isodose comparison of PRDR-IMRT and IMPT plans demonstrating how protons can reduce dose to uninvolved brain. FIG. 3 is a line graph showing a dose-volume histogram (DVH) comparison between PRDR-IMRT and PRDR-IMPT plans for a sample subject. FIGS. 4A-4D includes four line graphs comparing an uninvolved brain V10Gy, V20Gy, V30Gy and V40Gy for PRDR photon and PRDR-IMPT plans as a function of target (PTV) size, which show that for larger targets, IMPT plans provide a dose sparing advantage over photons, especially for target volumes >300 cc. FIGS. 5A and 5B are histograms showing minimum local and global gamma analysis for the six test cases with 3%/3 mm, 2%/2 mm, and 1%/1 mm analysis criteria, in which the 3%/3 mm global gamma analysis is >93% for repainted and non-repainted fields. FIG. 6 is a table showing a global gamma analysis for repainted and non-repainted cases evidencing that the 3%/3 mm global gamma analysis is >93% for repainted and non-repainted fields, which meets clinical treatment criteria.

In Example 1.3, as shown in FIG. 1A, a three (3) field CNS treatment with 1.8 Gy/Fx, was re-painted once resulting in six sub fields with each sub-field delivering approximately 0.3Gy/fx. Adjusting the time delay between each field (5-7 min) was found to allow for an overall dose rate of 7 cGy/min to be achieved. The total treatment time with image guidance was approximately 45 minutes.

### Results

Utilizing IMPT-PRDR, the estimated overall fractional treatment time, including imaging and patient setup, was approximately 1 hour. The key driving factors for this treatment time was the gaps between fields and the sub-beam repainting. IMPT-PRDR, treatment plans were dosimetrically compared to IMRT-PRDR treated cases and normalized to ensure comparable coverage to the PTV, and this is reflected by the CTV D99 being within 1% for 4 initial planned cases. Median average normal brain dose for the IMRT-PRDR plans was 16.6 Gy (Range: 14.6 Gy to 28.0 Gy), while for the IMPT-PRDR plans was 13.0 Gy (Range: 8.2 Gy to 16.4 Gy). On average, the IMPT-PRDR plan resulted in a 60% reduction (p=0.06) to normal brain dose, which is clinically significant, especially considering the re-irradiation nature of these cases.

TABLE 1

| | | V30 | V20 | | |
|---|---|---|---|---|---|
| | Case | Uninvolved | Uninvolved | Brainstem | Chiasm |
| | Number | Brain (%) | Brain (%) | D_{0.03cc} (Gy) | D_{0.03cc} (Gy) |
| Photon- | 1 | 0.19 | 11.28 | 0.69 | 1.88 |
| Proton | 2 | −0.41 | 3.57 | 3.68 | 10.34 |
| | 3 | 7.49 | 9.14 | 8.11 | 7.32 |
| | 4 | 0.71 | 2.67 | 6.01 | 13.75 |
| | 5 | 13.24 | 27.23 | −0.49 | 0.86 |
| | 6 | 7.15 | 29.88 | −1.45 | 3.44 |

### CONCLUSIONS

Examples 1.1 to 1.3 illustrate a methodology for delivering IMPT-PRDR in combination, which combines both the radiobiological advantages of low dose rate therapy and the integral dose sparing of proton therapy. It is believed that these benefits could be employed in clinical trials in the treatment of CNS cases that have undergone prior radiotherapy with no technical modifications to the IMPT system.

The present inventors have also recognized that a PRDR-IMPT treatment strategy allows for the delivery of an averaged effective treatment dose rate of approximately 7 cGy/min over one treatment fraction, which is similar to photon PRDR. Further, the inventors have recognized that PRDR-IMPT plans offer dosimetric sparing of uninvolved brain (V20Gy) and reduction in the D_{0.03cc} to the brainstem and optic chiasm when compared to photons. Further, it was determined that measurements of the repainted IMPT fields showed <0.5% change in the global gamma analysis for 3%/3 mm criteria, when compared to non-repainted IMPT fields, as shown in FIGS. 5A-6.

### Example 2

### Methods and Materials

### Data Acquisition

For Example 2, five (5) patients were treated with TMPPR. The patients who underwent the course of TMPPR for recurrent or progressive CNS malignancies were evaluated. All patients were evaluated in a multidisciplinary CNS tumor conference and selected for this option only when all conventional and clinical trial options were exhausted. Data collected from the electronic medical records included gender, age, tumor histology, Karnofsky Performance Status (KPS) at time of TMPPR, the number and dates of prior interventions, radiotherapy dose and fractionation schedule, and toxicities during treatment and follow-up.

Median age was 54 years (Range: 32-72), and median time from initial radiotherapy course to re-RT was 23 months (Range 14-40). The median dose was 60 GyRBE in 30 fractions and was completed without any delay in all patients. At first treatment response assessment, the best objective response rate was complete (CR) (n=1) or partial (PR) (n=3) response in four patients. Limited toxicity was seen. In particular, CTCAE grade 2 alopecia was observed in all patients and one case of radiation necrosis grade 2.

### Temporally Modulated Pulsed Proton Re-Irradiation (TMPPR) Workflow

### a) Planning and Treatment Simulation Imaging

At simulation, patients were placed in a supine position and a computerized tomography (CT) scan was performed for treatment planning using 1-mm slice thickness. Patient was immobilized using a thermoplastic mask (Fiberplast®, QFix, Avondale, PA) mask, a custom head rest (MoldCare®, QFix, Avondale, PA), and a knee cushion with hands holding pegs along their side or a ring across their chest for reproducibility and comfort. Diagnostic magnetic resonance (MRI) including a T1 post-Gadolinium (Gd) and a T2-weighted fluidattenuated inversion recovery (T2/FLAIR) sequences were also performed and co-registered to the treatment planning CT scan for target volume delineation purposes.

### b) Target Volumes and Organs-at-Risk Determination and Contouring

Planning and simulation images (MR and CT images) were co-registered in the treatment planning system (TPS)—RayStation v.9A (RaySearch Laboratories, Stockholm, Sweden)—for delineation of target volume and organs-at-risk (OARs). For target volumes, the gross tumor volume (GTV) was defined as the T1 contrast enhanced lesion and resection cavity. For glioma patients, the FLAIR abnormality was also included in the GTV. The clinical target volume (CTV) was defined as a variable GTV expansion, from 0 cm (inclusion of FLAIR only) to 1.5 cm isotropic expansion, limited by anatomical barriers (bone, contralateral brain, brainstem, and optic tract). For OARs, the uninvolved brain (total brain minus the CTV), brainstem, optic chiasm, ipsilateral and contralateral optic nerves, ipsilateral and contralateral cochlea, and ipsilateral and contralateral hippocampus were contoured.

### c) Treatment Planning and Delivery

For the TMPPR technique, reRT was planned and delivered using intensity-modulated proton therapy (IMPT) and a modified single field optimization (SFO) approach with 3 fields, where 70% of the total dose was controlled by each beam and the remaining 30% modulated for OAR sparring. Plans were robustly optimized to the CTV and evaluated using 3.5% range uncertainty and 3 mm setup uncertainty on a 2 mm dose grid. Beam angle selection was performed to ensure a large hinge angle among the three fields and at most only one of the three fields end ranging into an OAR. Each field was re-painted once, resulting in six equally weighted sub-fields with each delivering approximately a maximum dose of 0.3 Gy/fraction to the CTV. Beam maximum dose contribution was capped at 0.4Gy per fraction to limit modulation. Adjusting for the time delay among each field (5 min) yielded an overall time-averaged effective dose rate of <7 cGy/min

All CT datasets, dose distributions, and structure sets from previous radiotherapy courses were imported into the RayStation TPS to generate composite dose distributions for dosimetric evaluation. After generation of the composite does distributions, the cumulative equivalent dose in 2-Gy fractions (EQD2) distributions to each OAR were assessed for prior and current treatment plans. The following dose parameters were subsequently extracted: average dose, maximum dose to 0.03 cc, maximum dose to 0.5 cc, and maximum dose to 1 cc in EQD2 values for OARs, including the brain (excluding the CTV), brainstem, optic chiasm, ipsilateral and contralateral optic nerves, ipsilateral and contralateral cochlea, and ipsilateral and contralateral hippocampus, as well as the target volumes

For daily positioning, a cone-beam CT (CBCT) was performed and aligned to the reference CT for an accurate and precise administration of the dose to the target volumes. Patient position corrections were enabled using a 6 degree of freedom couch. The proton treatment was delivered by Proteus® PLUS Proton Therapy accelerator (Ion Beam Applications S.A.). The delivery time for each subfield and subsequently delay was annotated and documented in the treatment records for each fraction.

### Treatment Monitoring and Follow-Up

Weekly monitoring of acute toxicities occurred during treatment. Follow-up evaluations were conducted 4-6 weeks after radiotherapy completion, followed by subsequent assessments every 2-3 months. These evaluations involved clinical examination and the use of contrast enhanced brain MRI. Response Assessment in Neuro-Oncology (RANO) radiological response criteria were employed to assess treatment response, including complete response (CR), partial response (PR), stable disease (SD), or progressive disease (PD). Toxicity grades were assessed based on the criteria outlined in the National Cancer Institute CTCAE v5.0. Radiation necrosis was defined as the emergence or enlargement of contrast enhancement in the region previously treated with radiotherapy, excluding recurring tumors, and verified by evaluation of multi-parametric imaging and advanced options, such as MR perfusion. These cases were thoroughly discussed in a multidisciplinary tumor conference involving specialists from various fields, such as neuroradiology, neurosurgery, neuro-oncology, and radiation oncology.

### Pattern of Recurrence by Localization

Dose-Volume Histogram (DVH) was used to determine the spatial relationship between the recurrent tumor volume and the delivered dose distribution (prior irradiation). The DVH analysis enabled classification of recurrences into different categories based on their location. If more than 95% of the recurrence volume was found within the original high-dose field according to the DVH, it was categorized as a central failure. Recurrences with a distribution of more than 80% to 95%, 20% to 80%, and less than 20% were designated as in-field, marginal, and distant recurrences, respectively.

### Neurocognitive Evaluation

Patients were also enrolled onto an ongoing neurocognitive prospective observational registry (NCT05504681). To assess neurocognition, patients utilized a tablet-based app called the Brainlab Cognition app (Brainlab AG. Munich, Germany). The app presented patients with a comprehensive set of questions designed to evaluate various aspects of neurocognitive function. These questions encompassed measures of learning and memory, involving verbal recall, revision, and recognition testing (for immediate recall, delayed recall, and delayed recognition, respectively). Additionally, the app assessed attention and processing speed using symbol matching tests, verbal fluency with “words that start with” exercises, fine motor skills, speed with numbers ordering tests, and executive functions through numbers and letters ordering tests.

### Statistical Analysis

Descriptive statistics were computed. For continuous variables, the median and range were presented. Sample sizes and percentages were computed for categorical variables.

### Results

### Patient's Characteristics

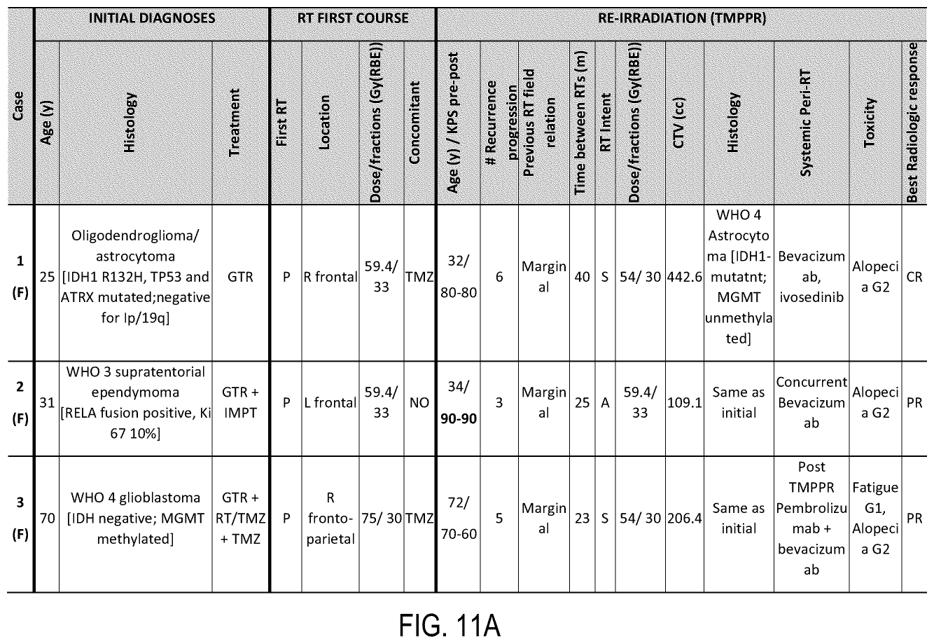

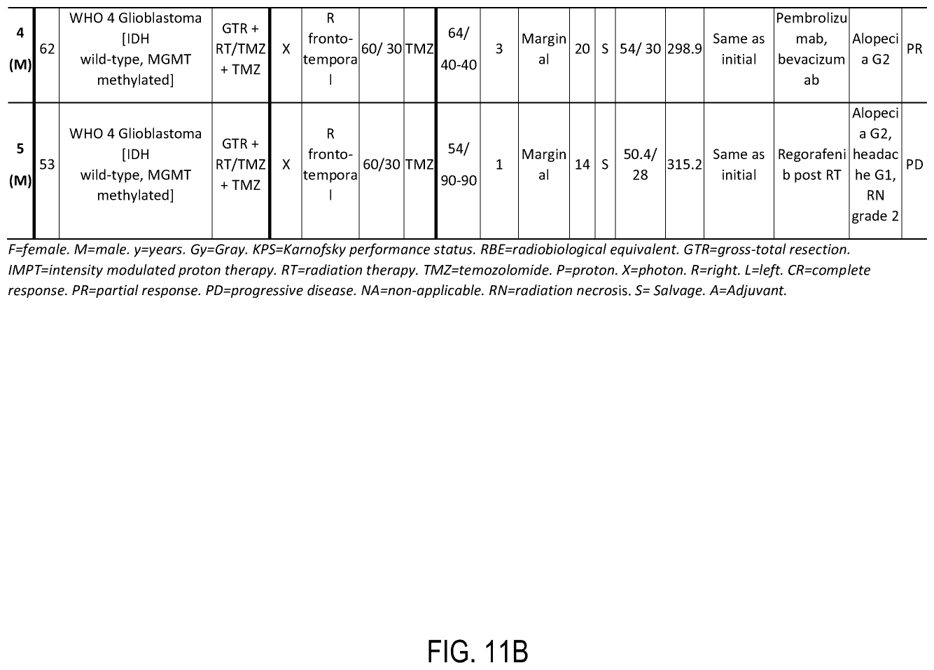

The first five consecutive patients treated with TMPPR for recurrent high-grade intracranial malignancies are included in this analysis, as shown in the tables in FIGS. 11A and 11B. The median age was 54 years (range: 32-72 years), and 3 patients (60%) were females.

The first course of radiation was delivered with proton-based RT for three cases and photon-based RT for two cases, with a median dose and dose per fraction of 60 Gy(RBE) (range: 59.4-75 Gy[RBE]) and 2 Gy(RBE) (range: 1.8-2.5 Gy[RBE]), respectively. Four patients received concurrent temozolomide (TMZ). The locations were solely frontal in two cases, two frontotemporal, and one frontoparietal. Four lesions were right-sided, and one left-sided. The most common histology for prior reRT was WHO grade 4 glioblastoma (IDH wildtype; MGMT methylated) in three patients, followed by one with WHO grade 4 astrocytoma (IDH mutated; MGMT unmethylated), and one with WHO grade 3 supratentorial ependymoma.

### Reirradiation Characteristics (TMPPR)

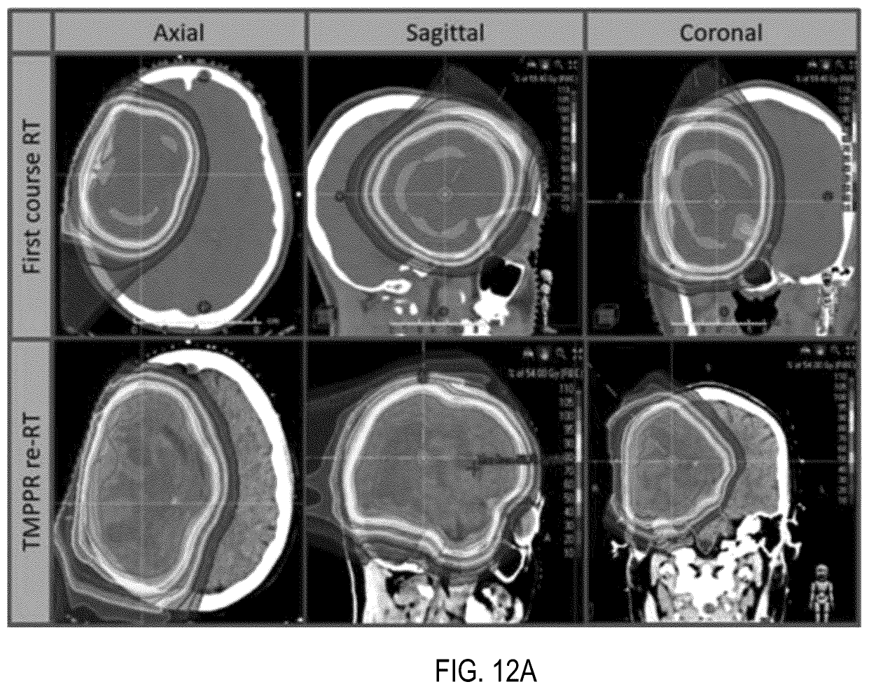

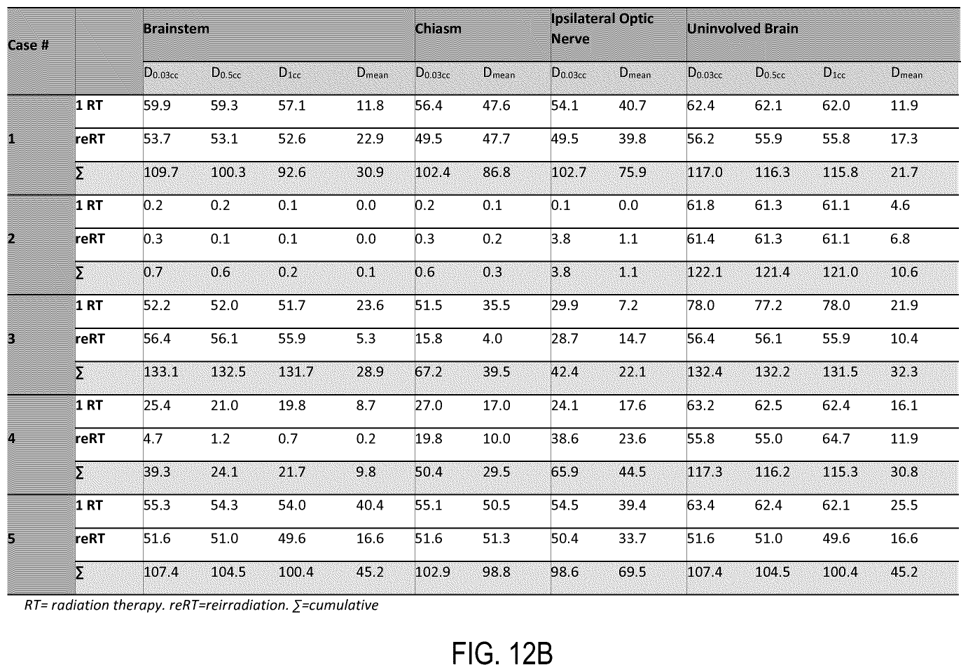

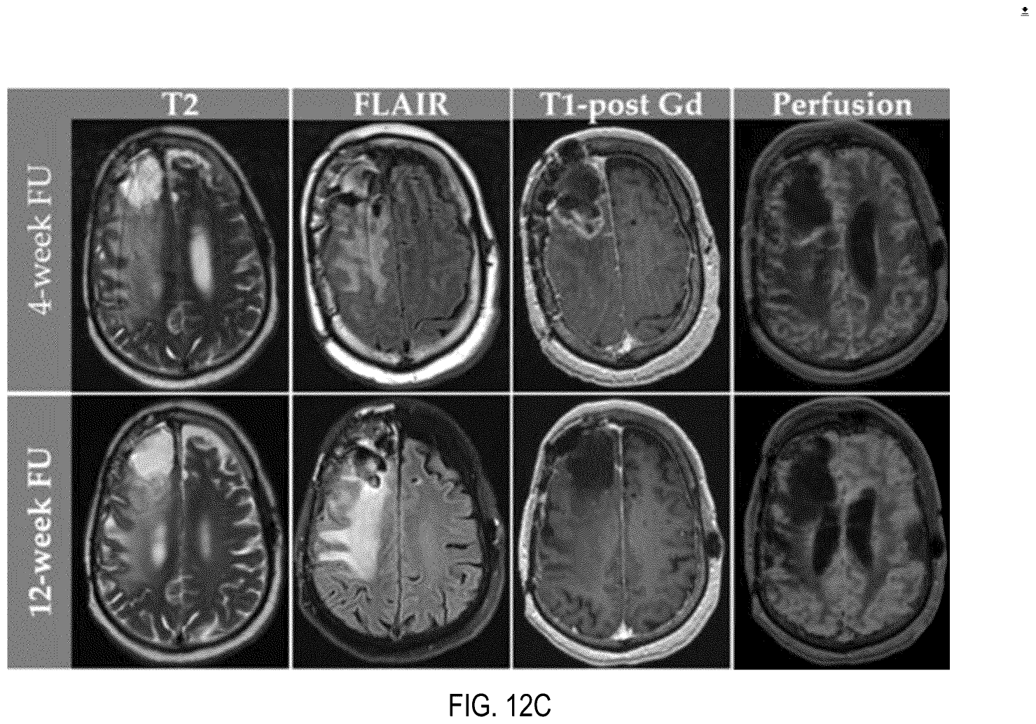

All patients had at least 1 recurrence prior to TMPPR (median 3 recurrences, range: 1-6) for which they underwent to one prior course of radiotherapy, at least one surgery (median: 3, range: 1-4) and one line of systemic therapy (median: 2, range: 1-4). The location of recurrence/progression was marginal to the prior RT field (since the recurrence volume within the high-dose field from previous radiotherapy was between 20-80%) in all the 5 patients, as shown by the medical images in FIG. 12A. The local treatment was TMPPR alone in 4 cases and TMPPR following surgical resection in 1 case. The median CTV was 298.9 cc (range: 109.1-442.6 cc), with a median total dose of 54 Gy(RBE) (range: 50.4-59.4 Gy[RBE]) in a median of 30 fractions (range: 28-33). All five patients tolerated TMPPR treatment without any delay or complication.

All patients had at least one MR follow-up scan after TMPPR completion. The MRI of one of the patients was required 10 days after the end of TMPPR treatment, due to the presence of worsening neurologic symptoms compatible with progression, in central location to the TMPPR. This patient was diagnosed with progressive disease and was referred to a hospice care facility.

The TMPPR course doses and cumulative doses received by the OARs are described in the table in FIG. 12B. The brainstem received median cumulative doses for D_{0.03cc}, D_{0.5cc}, D_{1cc}, and Dₘₑₐₙ of 107.4 Gy(RBE) (range: 0.7-133.1), 100.3 Gy(RBE) (range: 0.6-132.5), 92.6 Gy(RBE) (range: 0.2-131.7), and 28.9 Gy(RBE) (range: 0.1-45.2), respectively. For the uninvolved brain, the median cumulative doses for D_{0.03cc}, D_{0.5cc}, D_{1cc}, and Dₘₑₐₙ were 117.3 Gy(RBE) (range: 107.4-132.4), 116.3 Gy(RBE) (range: 104.5-132.2), 115.8 Gy(RBE) (range: 100.4-131.5), and 30.8 Gy(RBE) (range: 10.6-45.2), respectively. For the optic chiasm, the median cumulative doses for D_{0.03cc} and Dₘₑₐₙ were 67.2 Gy(RBE) (range: 0.6-102.9) and 39.5 Gy(RBE) (range: 0.3-98.8), respectively. Finally, for the ipsilateral optic nerve, the median cumulative doses for D_{0.03cc} and Dₘₑₐₙ were 65.9 Gy(RBE) (range: 3.8-102.7) and 44.5 Gy(RBE) (range: 1.1-75.9), respectively.

Daily fractions consisted in the delivery of three fields, each one subdivided in two subfields, with a total of six subfields. The approximated total time for each daily-fraction was 39.5 minutes, as shown by the schedule in FIG. 1B. Initial positioning and imaging time was 10 minutes; considering the prolonged time of each fraction, an interim imaging for positioning reassurance was obtained between the third and fourth subfield delivery (overlapping with the 3.5 minutes of waiting time). Beam-on time for each subfield was approximately 2 minutes (total beam-on time of 12 minutes). Finally, the wait time between each beam-on time per sub-fraction delivered was 3.5 minutes (total of 17.5 minutes).

### Clinical Outcomes and Treatment-Related Toxicity

All patients tolerated the TMPPR treatments well. Best objective radiological response consisted of one complete response (CR), three partial responses (PR), and one progressive disorder (PD). The first patient had the scan, while progressing on bevacizumab and ivosidenib and no corticosteroids, revealing a dramatic resolution of the large multi-lobulated enhancing mass, substantial reduction in the infiltrating T2/FLAIR signal, resolution of mass effect on the right lateral ventricle, and absence of elevated relative cerebral blood volume (rCBV) on the perfusion MR (which was previously elevated). These imaging features were consistent with a complete response to treatment.

All of the patients (100%) developed grade 2 alopecia (one with associated grade 1 fatigue, and other with headache grade 1). In one case, symptomatic radiation necrosis grade 2 occurred. This patient received bevacizumab with subsequent radiographic improvement on second follow-up MRI (12-week post-TMPPR) compared to the first follow-up MRI (4-week post-TMPPR), as shown in the images in FIG. 12C.

Three patients consented for enrollment in the prospective observational neurocognitive function registry. One had the baseline evaluation, but did not complete neurocognitive evaluation after treatment and the other patient continued follow-up with telemedicine visits only. One patient completed both baseline and follow-up evaluations demonstrating stability of neurocognitive function across multiple tested domains, as displayed in the graph in FIG. 8, which was described previously in this disclosure.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment

The invention claimed is:

1. A method for re-treatment of a tumor, comprising:
providing therapy pulses to the tumor in accordance with a pulsed reduced dose rate (PRDR) therapy pattern,
wherein the therapy pulses are PRDR intensity modulated proton therapy (IMPT) pulses comprising proton pulses provided to the tumor.

2. The method of claim 1, wherein the tumor was previously treated by applying pulsed reduced dose rate (PRDR) photon therapy pulses to the tumor.

3. The method of claim 2, wherein the PRDR photon therapy pulses are about 0.2 Gy/pulse (2 Gy/fx) with an interval of at least 3 minutes between the PRDR photon therapy pulses.

4. The method of claim 1, wherein the PRDR-IMPT pulses have an averaged effective treatment dose rate of approximately 7 cGy/min over one treatment fraction.

5. The method of claim 1, wherein there is a temporal gap of about 4 minutes to about 5 minutes between the PRDR-IMPT pulses.

6. The method of claim 1, wherein the PRDR-IMPT pulses are provided to repaint a treatment field previously treated by PRDR photon therapy pulses.

7. The method of claim 1, wherein the tumor comprises a central nervous system (CNS) tumor and/or an intracranial tumor.

8. The method of claim 1, wherein the pulsed reduced dose rate (PRDR) therapy pattern comprises delivery of a dose of about 50 GyRBE to about 75 GyRBE divided between about 20 fractions to about 40 fractions.

9. The method of claim 8, wherein the fractions are delivered to a patient over a period of time of about 20 minutes to about 50 minutes.

10. The method of claim 1, wherein at most about 70% of a total dose of proton therapy pulses delivered to the tumor is controlled by each beam of an ion beam applicator and a remaining at least 30% of the total dose is modulated for organs-at-risk (OAR) sparring.

11. The method of claim 1, wherein the therapy pulses are provided according to one or more of the following therapies: stereotactic radiosurgery (SRS), fractionated SRS, hypofractionated treatment, fractionated proton therapy, or particle therapy.

12. The method of claim 1, wherein providing the therapy pulses comprises directing a radiation beam to at least one of the following areas of a patient's body: brain, brainstem, optic chiasm, ipsilateral and/or contralateral optic nerves, ipsilateral and/or contralateral cochlea, or ipsilateral and/or contralateral hippocampus.

13. The method of claim 1, further comprising administering an effective amount of at least one of bevacizumab and ivosidenib to a patient for treatment of the tumor.

14. A system for re-treatment of a tumor, comprising:
a first sub-beam emitter for providing a first plurality of intensity modulated proton therapy (IMPT) pulses in accordance with a pulsed reduced dose rate (PRDR) therapy pattern;
a second sub-beam emitter for providing a second plurality of intensity modulated proton therapy (IMPT) pulses in accordance with the PRDR therapy pattern; and
a controller electrically connected to the first and second sub-beam emitters, wherein the controller is configured to cause the first plurality of IMPT pulses and the second plurality of IMPT pulses to be provided to the tumor sequentially with a temporal gap between delivery of one or more of the first plurality of IMPT pulses and one or more of the second plurality of IMPT pulses.

15. The system of claim 14, wherein the controller is configured to cause the first sub-beam emitter and the second sub-beam emitter to provide PRDR-IMPT pulses having an averaged effective treatment dose rate of approximately 7 cGy/min over one treatment fraction.

16. The system of claim 14, wherein the temporal gap between the delivery of one or more of the first plurality of IMPT pulses and one or more of the second plurality of IMPT pulses is from about 4 minutes to about 5 minutes.

17. The system of claim 14, wherein the tumor is a central nervous system (CNS) tumor and/or an intracranial tumor.

18. The system of claim 14, wherein at most about 70% of a total dose provided by the first sub-beam emitter and the second sub-beam emitter is controlled by the sub-beam emitters, and a remaining at least 30% of the total dose is modulated for organs-at-risk (OAR) sparring.

19. The system of claim 14, wherein the controller is configured to cause the first and second sub-beam emitters to deliver the first and second plurality of IMPT pulses for a period of time of about 20 minutes to about 50 minutes in order to provide a total dose of the IMPT pulses to the tumor.

20. The system of claim 14, wherein the controller is configured to cause the first sub-beam emitter and the second sub-beam emitter to deliver the pulsed reduced dose rate (PRDR) therapy pattern, which comprises delivery of a total dose of from about 50 GyRBE to about 75 GyRBE divided over about 20 fractions to about 40 fractions.
